(19) United States
(12) Patent Application Publication
(10) Pub. No.: US 2006/0122563 A1
(43) Pub. Date: Jun. 8, 2006

(54) Applicator assembly for application of substances to a body orifice and cavity

(76) Inventor: Elan Ziv, Ramat Gan (IL)

Correspondence Address: NATH & ASSOCIATES, 112 South West Street, Alexandria, VA, 22314, US

(21) Appl. No.: 11/199,376

(22) Filed: Aug. 9, 2005

Related U.S. Application Data
(60) Provisional application No. 60/599,577, filed on Aug. 9, 2004.

(51) Int. Cl. A61M 5/00

(52) U.S. Cl. 604/187

(57) ABSTRACT

The present invention concerns an applicator arrangement and a syringe equipped with said applicator arrangement, for delivery of fluid from the lumen of the syringe's to an interior and lips of a body cavity. The applicator arrangement comprises a tubular body having a proximal end and a distal end spaced apart along the tubular body axis and an interior passage for fluid transfer therein, the tubular body being integrally formed at the distal end with a lip abutment skirt having a cross-sectional area greater than that of said body, wherein the tubular body and the skirt HAVE, each a plurality of apertures for delivery of fluid therethrough from the syringe's lumen to the body's cavity and lips.

This application claims benefit of U.S. Provisional Patent Application 60/599,577 filed Aug. 9, 2004, the contents of which are hereby incorporated by reference in its entirety.

### FIELD OF THE INVENTION

The invention concerns devices for application of substances such as drugs into and at the orifice of bodily cavities, such as the rectum and vagina.

### BACKGROUND OF THE INVENTION

There are several instances in medicine in which a drug or a substance has to be applied topically onto the inner lining of a hollow organ and the adjacent surrounding tissues. This may occur when treating the vagina and the vulva, the anus and the anal canal, the uterine cervical canal and the portio vaginalis, the nostrils, etc. One example of such an application is for treating vaginal dryness.

Vaginal dryness can be experienced by all women over the age of 18 at some point in their lives. While the largest group who experience vaginal dryness is among menopausal women, there are many other factors that affect or increase the problem. Some, but by no means all, are related to lower estrogen levels from menopause (peri- to post-), menstrual cycle changes, pregnancy, childbirth, nursing, some hormonal contraceptives, endometriosis drugs, infertility drugs, hysterectomy and other related surgeries, stress, fatigue, rigorous exercise, intercourse, diaphragms, antidepressants, antihistamines, decongestants, antibiotics, chemotherapy, radiation, drying soaps, douches, tampons, condoms, etc.

Sufferers of vaginal dryness complain of having the feeling of constant or temporary dryness within the lower third of the vagina, and around the vulvar area. They may also note itching, burning, and pains during intercourse. Oral treatment with estrogen replacement therapy may reduce the symptoms to some extent but will not abolish them altogether, leaving about 15% of the patients with their complaints.

Current local treatments may be divided into two: treatment using lubricants or anesthetics in order to relieve the unpleasant sensation, thereby dealing with the complaint itself; or treatment using estrogen cream in order to reduce tissue atrophy, thereby dealing with the underlying cause of the symptoms.

Hitherto, application of substances into a body cavity included either digital application by using one or more fingers of the hand in order to apply the substance into the part of the cavity adjacent to the orifice or by the use of applicators.

Applicators are typically in the form of a syringe having a barrel and an nozzle. U.S. Pat. No. 5,843,043 describes one such syringe having a removable, tapered nozzle with side openings through which a treatment substance can be expelled against the wall defining a body cavity.

### SUMMARY OF INVENTION

Some medical conditions require application of medicaments to part of a body cavity proximal to the orifice and to and the outer surroundings (lips) of the orifice. Thus, according to a first aspect, the present invention provides an applicator assembly for delivery of fluid to an interior and lips of a body cavity comprising a tubular body having a proximal end and a distal end spaced apart along a tubular body axis and an interior passage for fluid transfer therein, the tubular body being integrally formed at the distal end with a lip abutment skirt having a cross-sectional area greater than that of the tubular body, and said lip abutment skirt having a side wall integrally formed at one end of a barrel or configured to be mounted onto one end of a barrel for fluid communication between said barrel and said interior passage of the tubular body, wherein said tubular body has a wall and said lip abutment skirt has an top, and each has multiple apertures for passage therethrough of fluid from the barrel towards the interior and lips of the body cavity.

The term “body cavity” as used herein denotes any body cavity having an orifice on the external surface of the body. In a typical mammalian body such as the human body, the body orifices include the nostrils, the mouth, the anus, the urethra, the uterine cervix canal, and in females, the vagina. According to a preferred embodiment, the body cavity and orifice includes the anus and vagina.

The term “interior of a body cavity” denotes the portion of the cavity proximal to its orifice, preferably first third from the lips, of the cavity. The interior of the body cavity is defined by a cavity and walls defining the cavity's boundaries. The cavity's boundaries define the cross-sectional area of the orifice, which is typically larger than the cross-sectional area of the tubular body forming part of the applicator arrangement of the invention. When inserted into the interior of the cavity, the walls of the tubular body face the walls of the cavity such that, upon operation, fluid is ejected radially from the apertures in the tubular body's wall in a lateral direction, towards the walls of the cavity. The tubular body is elongated and has a length so as to enable the ejection of fluid to the part of the body cavity proximal to its orifice. According to a preferred embodiment, the fluid is evenly ejected through said apertures.

The term “lips of a body cavity” denotes the folds of the skin surrounding the orifice of the cavity. With respect to the vagina, lips denote the labia majora and labia minora.

The term “fluid” according to the invention preferably denotes any liquefied substance typically applied to body cavity via the body cavity's orifice, e.g. gel, cream, ointment, etc., having various degrees of viscosity.

The invention also provides a syringe for ejecting a selected amount of a fluid substance to an interior and lips of a body cavity comprising a barrel having at its end the applicator arrangement of the invention.

### BRIEF DESCRIPTION OF THE DRAWINGS

In order to understand the invention and to see how it may be carried out in practice, exemplary embodiments will now be described, by way of non-limiting examples only, with reference to the following accompanying drawings.

FIGS. 1A-1C are schematic illustrations of a applicator arrangement for delivery of fluids according to one embodiment of the invention, including a partial cutaway longitudinal cross-sectional view (FIG. 1A), a perspective view (FIG. 1B) and an upper elevational view (FIG. 1C) thereof.

FIGS. 2A-2D are partial cutaway perspective views of applicator arrangements according to alternative embodiments of the invention.

FIG. 3 is a schematic longitudinal cross-sectional view of an applicator arrangement according to the invention, integrally formed with a barrel of a syringe component.

FIG. 4 is a partial cutaway longitudinal cross-sectional view of an applicator arrangement according to another embodiment of the invention when mounted on a barrel by friction engagement.

FIG. 5A is a partial cutaway cross-sectional view of a front end of a substance-containing cartridge to be inserted in a barrel in accordance with one embodiment of the invention.

FIGS. 5B-5C are partial cutaway cross-sectional (FIG. 5B) and upper elevational (FIG. 5C) views of an applicator arrangement region of a syringe when loaded with a cartridge, the syringe being equipped with cutting blades in accordance with an embodiment of the invention.

### DETAILED DESCRIPTION OF EXEMPLARY EMBODIMENTS

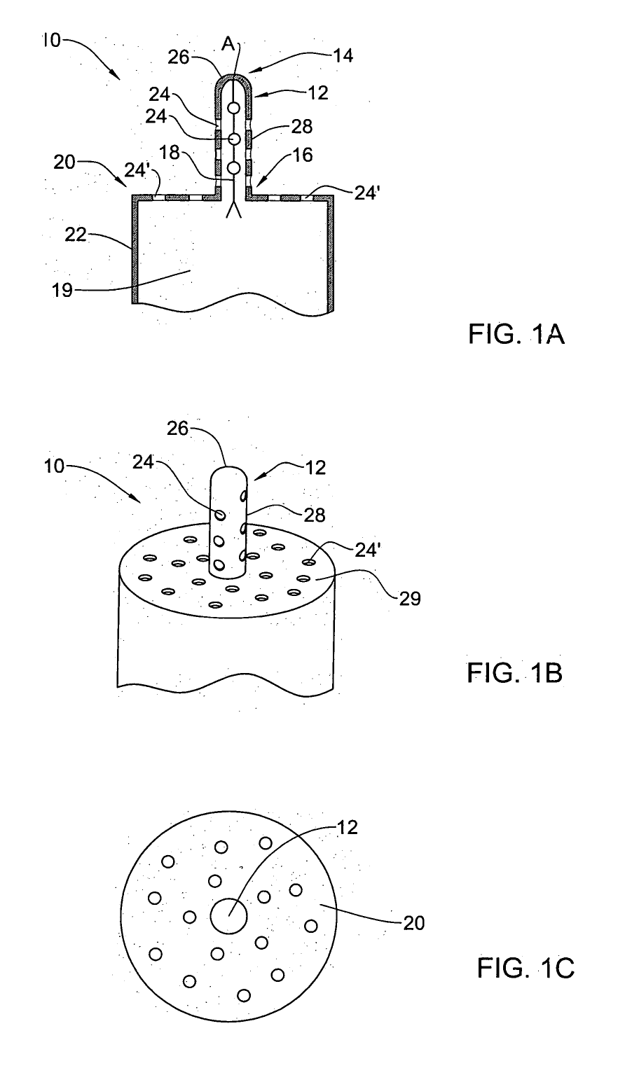

Reference is now made to FIG. 1A, which is a cross-sectional view of an applicator arrangement for delivery of fluid to an interior and lips of a body cavity according to one embodiment of the invention. The applicator arrangement 10 comprises an elongated tubular body 12 having a sealed proximal end 14 and a distal end 16 spaced apart along the tubular body axis A, a wall 28 and an interior passage 18 communicating with the barrier 19 of barrel 22. The tubular body's wall 28 at distal end 16 flattens or extends out to form a lip abutment skirt 20, which extends radially outward from the periphery of the tubular body 12, thereby limiting the insertion of body 12 into a body cavity. The lip abutment skirt 20 has a cross-sectional area greater than that of the tubular body 12. The lip abutment skirt, in this embodiment, is integrally formed of an end of barrel defined by side wall 22. It is understood, that rather than integrally formed in the manner described, it is also possible, under other embodiments of the invention to configure the applicator arrangement for monitoring at an end of a barrel of a syringe. As also illustrated in FIGS. 1B and 1C, a plurality of apertures 24 and 24′ are formed in the walls of tubular body 12 and in the skirt 20, respectively.

As shown above the tubular body's 12 front end 26 is sealed, to prevent expulsion of fluid farther along the tubular body's longitudinal axis A into the body cavity. According to this embodiment, the front end 26 has generally a hemispherical shape with no outwardly facing sharp points or edges so as to avoid inconvenient scraping of the walls of the body orifice and cavity, when inserting the applicator arrangement via the cavity's orifice.

While in the specific embodiment illustrated in FIGS. 1A-1C the shapes of the cross-section of the tubular body 12 and the skirt 20 are circular, it is to be understood that the applicator arrangement may each, independently, similarly function with an oval or other suitable cross-sectional shape. According to one embodiment, the walls 28 of tubular body 12, as well as the walls 22 of skirt 20, each have a relatively uniform thickness.

As indicated above, there are a plurality of apertures 24 in the wall of tubular body 12 and a plurality of apertures 24′ in the skirt 20. According to the invention the term “plurality of apertures” denotes two, preferably more, apertures of any shape in the wall of the tubular body and in the skirt. The apertures may be, e.g., circular, oval, and star-like shaped; the apertures may be all the same or may have different shapes or sizes; they may be randomly distributed or have a specific distribution pattern, e.g. in radially, longitudinally and/or diagonally arranged lines or in a sprinkle-like arrangement. The presence of apertures in the wall of the tubular body and the top of the lip abutment skirt is a unique feature of the invention ensuring that a substance dispensed laterally through the tubular body's apertures and upwardly through the skirt's apertures is applied, respectively, to a lower part of the body cavity (i.e. the area proximal to the cavity's orifice) and to the lip of a body orifice, i.e. surroundings of the opening of the cavity.

The applicator arrangement according to the invention may be made of a rigid or flexible material. Preferably, the applicator arrangement is made of a relatively flexible material so as to reduce the chances of inadvertent injury to the body cavity. It is also preferable that the skirt (or a part thereof) is made of a rigid or semi-rigid material (e.g. plastic) so as to enable support of the applicator arrangement against the lips of the cavity's orifice, while pressing a substance through the interior passage and through the apertures.

According to one preferred embodiment, the applicator arrangement is made from a smooth disposable material, such as polyethylene, polycarbonate, other plastics, silicone or of cardboard.

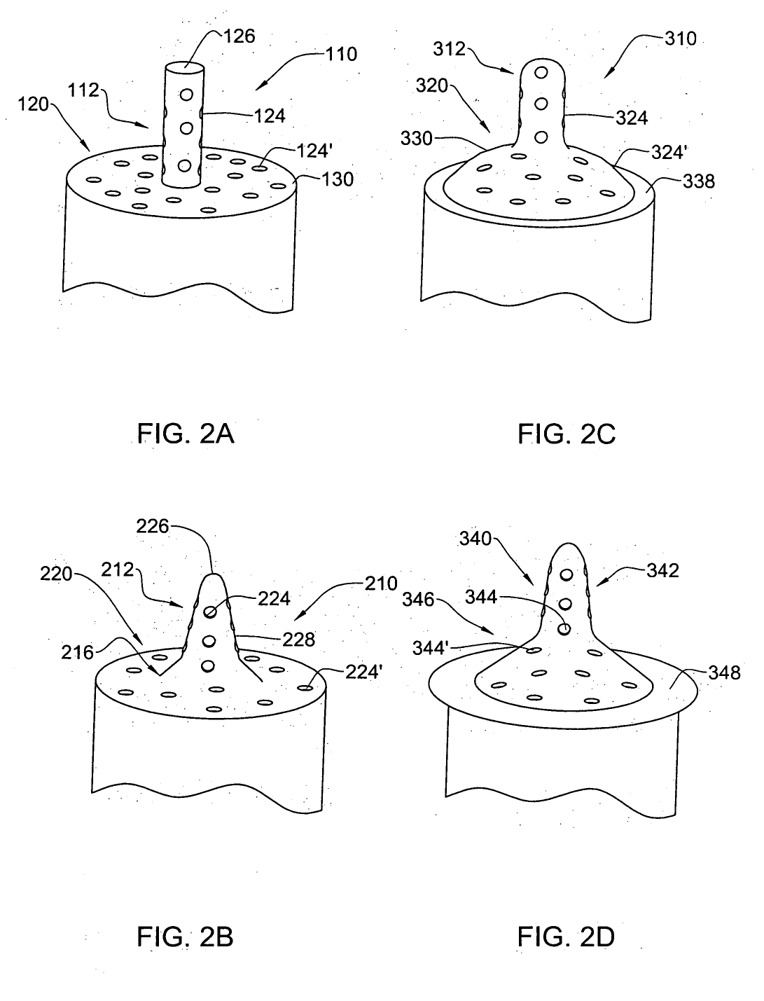

Reference is now made to FIGS. 2A-2C, which represent alternative embodiments of the applicator arrangement illustrated in FIG. 1B. Thus, elements which are identical to those illustrated in FIGS. 1A-1C are marked in FIGS. 2A-2C with the same index numbers, however, increased by a hundred, two hundred and three hundred, respectively.

FIG. 2A illustrates an applicator arrangement 110 having a tubular body 112 and a lip abutment skirt 120. The tubular body 112 has a plurality of apertures 124 and the skirt 120 has a plurality of apertures 124′. Tubular body 112 has a flat front end 126.

FIG. 2B illustrates an applicator arrangement 210 having a tubular body 212 and a lip abutment skirt 220, each having, respectively, a plurality of apertures 224 and 224′. Tubular body 212 has a conical shape with a tapered wall 228 having a small, hemi-spherically rounded front end 226.

FIG. 2C illustrates an applicator arrangement 310 having a tubular body 312 carrying a plurality of apertures 324, and a skirt 320 with apertures 324′. The skirt 330 has a central portion which ha a general dome-shape and has a flat peripheral rim portion 338. As can be seen, in this embodiment, the apertures of the skirt 320 are confined to the central portion 330.

FIG. 2D illustrates an applicator arrangement 340 having a tubular body 342 carrying a plurality of apertures 344, and a lip abutment skirt 346 having a shape of a cone converging towards the tubular body 342. The skirt 346 also has a plurality of apertures 344′ and at its periphery defines an enforcement rim 348. The tubular body 342 extends upwardly from the top part of the skirt 346. The enforcement rim according to this embodiment is preferably made of rigid material, while the applicator arrangement may be made of a flexible or rigid material or a combination of a flexible and rigid material.

While tubular body 312 and the cone shaped part of the skirt 320 may be constructed from relatively flexible material to avoid discomfort at the points of contact with the walls of a cavity and lips of a body orifice, it is preferable that the enforcement rim 348 be constructed from a rigid or semi rigid material to provide support against any digital pressure applied during application of a substance into and at the surroundings of the body orifice.

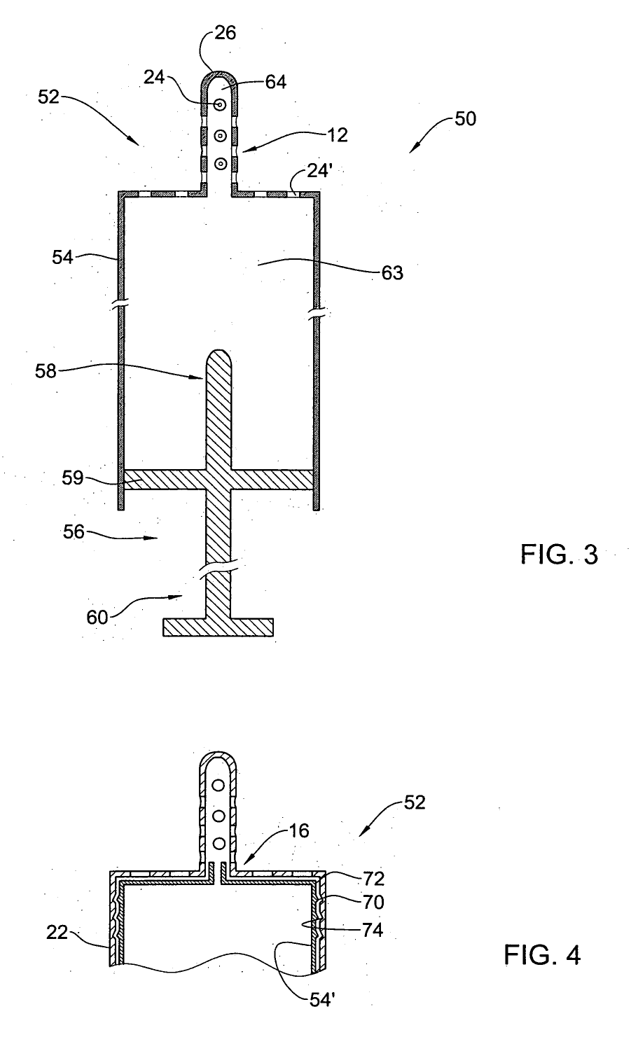

FIG. 3 illustrates a syringe 50 comprising an application arrangement 52, of the kind in FIGS. 1A-1C, a barrel 54 and a plunger 56. The applicator arrangement 52 is integral with the barrel 54. The barrel has a lumen 63 communicating with the lumen 64 of tubular body 12. The plunger 56 comprises a front part 58, a disk member 59 and a handle 60, movable longitudinally along the axis defined by barrel 54. The front part 58 has a shape so as to fit into the lumen 64 of tubular body 12. The handle 60 has a length which permits full advancement of the plunger along the barrel 54. Once fully advanced, the front part 58 of the plunger is shaped so as to fit into lumen 64, once the plunger is fully depressed, and leaves essentially no empty space that might retain fluid that should be dispensed through apertures 24 and 24′.

FIG. 4 provides an alternative construction of an applicator arrangement 52 according to the invention. According to this embodiment, the applicator arrangement 52 is mounted onto a barrel 54′ by means of friction engagement. To this end, the barrel 54′ has an external thread or corrugated surface 70 in its front end 72 and the applicator arrangement 52 has a matching internal thread or corrugated surface 74 in wall 22.

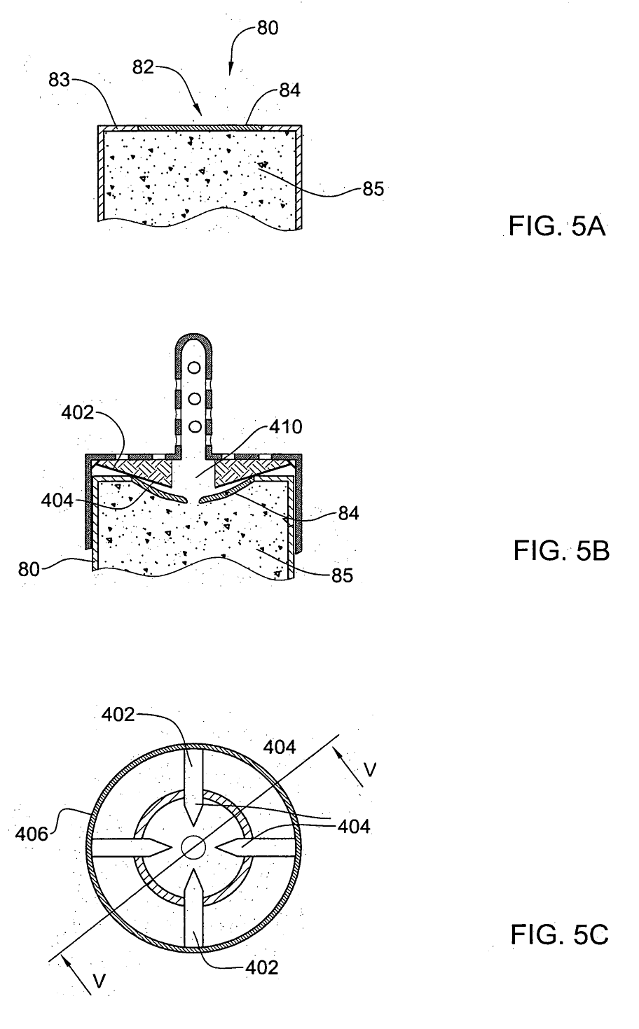

The barrel may be pre-filled with the substance and provided to the user as a ‘ready-to-use’ package. Alternatively, the user may be provided with a package comprising an empty (sterile) barrel and a cartridge comprising the substance. The cartridge is then inserted into the barrel before use. FIG. 5A is a cross-sectional view of an exemplary cartridge 80 to be mounted into a barrel such as that illustrated in FIG. 3 or 4. The cartridge has a general shape so as to fit into barrel 54 or 54′ (in FIGS. 3 and 4, respectively), with an opening 82 at the front end 83 thereof, to be positioned juxtaposed to the distal end 16 of the applicator arrangement 52 (e.g. of FIG. 3). The opening 82 is sealed with a membrane 84 which is punctured prior to use by suitable puncturing means. The membrane 84 may be made, for example, from nylon, aluminum foil or any suitable plastic which is pierceable, e.g. by cutting means present in the syringe, as will be explained below. The contents 85 of the cartridge may be a gelatinous substance, cream, ointment, etc. It should be appreciated by those versed in the art that the number and/or diameter and/or shape of apertures of the applicator arrangement will, inter alia, depend upon viscosity of the specific substance to be ejected therethrough.

FIG. 5B is a partial cutaway view along line V-V in FIG. 5C showing a cartridge 80, such as that described in FIG. 4, loaded into a syringe having an applicator arrangement similar to that illustrated in FIG. 1. The front end of the syringe is equipped with an internal cutting blade 402 having a cutting tip 404. When the cartridge 80 is fully inserted, the membrane 84 is punctured by the cutting tips 404. Applying pressure onto cartridge 80 e.g., by the syringe's plunger (not shown) will give rise to the ejection of substance 85 from the cartridge 80 into a space 410 remaining in the front of cartridge 80, and from there out through the apertures.

According to one embodiment, the applicator arrangement is constructed so as to enable the provision of a substance to a body cavity, preferably the vagina or anus, and lips thereof. In order to achieve a desired effect, the substance is applied onto the third part of the body cavity proximal to the orifice thereof, e.g. the lower third of the vagina, and on top of the vestibular mucosa and the inner side of the labias, preferably at equal quantities. To this end, the tubular body has a length not longer than the lower third of the vagina. It should be noted that digital application of a substance to the above three anatomical locations will typically not result in a uniform distribution of the substance to these location. Furthermore, women are typically reluctant to perform such a procedure even if being of therapeutic benefit.

Upon use, a syringe having an applicator arrangement of the invention is located such that the applicator arrangement is inserted into the vaginal orifice with the skirt being applied against the vestibule and the labia minora. Once in place the user applies pressure onto the cartridge within the barrel, resulting in the ejection of preferably essentially equal amounts of substance via the applicator arrangement's apertures. The entire content of the cartridge may be applied at a single use. Alternatively, the syringe may be equipped with slots defining dispensing volumes. To this end, it is preferable that the applicator arrangement is a disposable applicator arrangement, to be mounted onto the syringe in a manner such as that described with reference to FIG. 4.

Various conditions of the vagina or anus may be treated by the use of an applicator arrangement according to the invention. For example, the applicator arrangement may be used for the treatment of vaginal conditions selected from vulvo-vaginal dryness, dyspareunia, vulvo-vaginal infection, vulvo-vaginal itching vulvo-vaginal atrophy and hemorrhoids.

The substance to be applied to the vagina may be a pharmaceutical composition comprising, e.g., anesthetics, a lubricant, antibiotics, fungicides, steroids for topical application, a hormone supplement.

The applicator arrangement may also be used in a similar manner as described above for the application of a substance to the lower third of the alimentary canal (the rectum) and anus and may include, without being limited thereto: mild hemorrhoids, fissures, ulcerations, eczema, etc.

The substance in the barrel may vary. Non-limited examples include pharmaceutical compositions comprising anesthetics, lubricants, antibiotics, fungicides, steroids, hormone supplements, and combinations of same.

Yet further, the applicator arrangement may be similarly used for the application of substances to the nostrils, e.g. for preventing nasal dryness which may cause discomfort, soreness, congested breathing, and even bleeding and the associated possibility of nasal infections. To this end, the cartridge may be loaded with suitable hydrating gels together with a therapeutic agent suitable for the specific condition, as known to those versed in the art.

Those skilled in the art to which this invention pertains will readily appreciate that numerous changes, variations and modifications can be made without departing from the scope of the invention mutatis mutandis.

1. An applicator arrangement formed at an end of a barrel or adapted for mounting at a barrel end for delivery of fluid from the barrel to an interior and lips of a body cavity comprising a tubular body having a proximal end and a distal end spaced apart along the tubular body axis and an interior passage for fluid transfer therein, the tubular body being integrally formed at the distal end with a lip abutment skirt having a cross-sectional area greater than that of said body, said body and said skirt having a plurality of apertures for delivery of fluid therethrough

2. The applicator arrangement of claim 1, adapted for delivery of fluid to said lips and to a wall of a part of said body cavity proximal to said lips.

3. The applicator arrangement of claim 1, wherein said tubular body has a sealed front end.

4. The applicator arrangement of claim 1, having a plurality of apertures distributed in said applicator arrangement in a defined pattern.

5. The applicator arrangement of claim 1, wherein said tubular body is made of a flexible material and said lip abutment skirt is made of a rigid or semi-rigid material.

6. The applicator arrangement of claim 1, equipped with internal cutting means.

7. The applicator arrangement of claim 1, which upon operation evenly ejects fluid through the apertures in said tubular body and said lip abutment skirt.

8. A syringe for ejecting a selected amount of a fluid to an interior and lips of a body cavity comprising a barrel having at its end an applicator arrangement formed at an end of a barrel or adapted for mounting at a barrel end for delivery of fluid from the barrel to an interior and lips of a body cavity comprising a tubular body having a proximal end and a distal end spaced apart along the tubular body axis and an interior passage for fluid transfer therein, the tubular body being integrally formed at the distal end with a lip abutment skirt having a cross-sectional area greater than that of said body, said body and said skirt having a plurality of apertures for delivery of fluid therethrough

9. The syringe of claim 8, wherein said applicator arrangement is adapted for delivery of fluid to said lips and to a wall of a part of said body cavity proximal to said lips.

10. The syringe of claim 8, wherein said tubular body has a sealed front end.

11. The syringe of claim 8, having a plurality of apertures distributed in said applicator arrangement in a defined pattern.

12. The syringe of claim 8, wherein said tubular body is made of a flexible material and said lip abutment skirt is made of a rigid or semi-rigid material.

13. The syringe of claim 8, wherein said applicator arrangement is equipped with internal cutting means.

14. The syringe of claim 8, which upon operation evenly ejects fluid through the apertures in said tubular body and said lip abutment skirt.
